(19) United States
(12) Patent Application Publication
(10) Pub. No.: US 2020/0261351 A1
(43) Pub. Date: Aug. 20, 2020

(54) ANTIOXIDIZING, ANTIAGING, OR ANTI-INFLAMMATORY COMPOSITION FOR STRENGTHENING SKIN BARRIER AND CARING FOR SKIN CELL DAMAGE CAUSED BY FINE DUST INCLUDING FERMENTED TEA EXTRACT

(71) Applicant: AMOREPACIFIC CORPORATION, Seoul (KR)

(72) Inventors: Hyoung-June KIM, Yongin-si (KR); Kyeonghwan HWANG, Yongin-si (KR); Jun Seong PARK, Yongin-si (KR); Tae Ryong LEE, Yongin-si (KR)

(73) Assignee: AMOREPACIFIC CORPORATION, Seoul (KR)

(21) Appl. No.: 16/651,878

(22) PCT Filed: Aug. 23, 2018
(86) PCT No.: PCT/KR2018/009719

§ 371 Date: Mar. 27, 2020

(30) Foreign Application Priority Data
Sep. 29, 2017 (KR) 10-2017-0128159
Sep. 29, 2017 (KR) 10-2017-0128174
Sep. 29, 2017 (KR) 10-2017-0128175

(51) Int. Cl. A61K 8/9789; A61K 36/82; A23L 33/105; A61Q 19/08

(52) U.S. Cl. CPC A61K 8/9789; A61K 36/82; A61K 2236/19; A61Q 19/08; A23L 33/105

(57) ABSTRACT

The present specification discloses a composition for caring for skin damage caused by fine dust, wherein the composition includes a fermented tea extract as an active ingredient and adjusts the expression level of at least one selected from the following group of genes which are present in skin cells and have expression levels that are affected by fine dust to normal levels, said group consisting of: IL-1B (NM_000576), IL-36G (NM_019618), S100A7 (NM_002963), LCE3D (NM_032563), PTGS2 (NM_000963) and XDH (NM_000379). Damage to skin cells can be cared for by using the composition for caring for skin damage caused by fine dust to return gene expression levels, which change due to fine dust, to normal levels.

### TECHNICAL FIELD

Disclosed in the present disclosure is composition for enhancing skin barrier. More specifically, disclosed is a composition comprising a fermented tea extract, which treats skin cell damage by significantly changing the expression level of biomarkers, etc., which are skin cell genes the expression level of which is changed by fine dust as compared to skin cells in normal state, enhances skin barrier by significantly changing the expression level of biomarkers, etc., which are skin cell genes the expression level of which is changed by weakening of the skin barrier as compared to skin cells in normal state, or inhibits the oxidation, aging and inflammation of skin cells by significantly changing the expression level of biomarkers, etc., which are skin cell genes the expression level of which is changed by oxidation, aging and inflammation as compared to skin cells in normal state.

### BACKGROUND ART

Skin is a part of the body that is directly exposed to the external environment. It not only serves as a protective layer for protecting important organs of our body, but also regulates water evaporation and protects the body from external infections. However, even though the skin prevents viral invasion from the outside, excessive exposure of the skin to ultraviolet rays or pollutants causes skin irritation. Particularly, the skin is damaged by Asian dust which is accompanied by strong wind and dirt.

Asian dust is a phenomenon in which small-sized sands or red clays float from the inland deserts of China, Mongolia, etc., are carried far away by the upper winds and then drop near the ground. In Korea, Asian dust occurs periodically every spring. Asian dust is a combination of organic and inorganic materials, and its physical characteristics and constituents are very diverse depending on the time and place of occurrence. It also includes metals that can have biological effects. The larger-sized particles of Asian dust usually stay in or around their, and the smaller-sized particles thereof flow even into Korea. It is reported that, when inhaled, this dust is deposited in the lower bronchial tubes and even in the gas exchange part of the lungs, which may cause damage to the respiratory system. In addition, it was found that skin cell damage was increased in the skin of people living in areas with lots of Asian dust or dirt.

Among the layers of the skin, the epidermis plays an important role in preventing evaporation of water out of the human body. The epidermis is divided into the stratum corneum, the stratum granulosum, the stratum spinosum and the stratum basale from the outside. The cells of the stratum corneum act like bricks, and the intercellular lipids between keratinocytes act like mortar to constitute the skin barrier. In addition, the keratinocytes of healthy people have natural moisturizing factors (NMF) at high concentrations, which help to retain moisture in the skin. For example, water-soluble substances such as amino acids effectively combine with water and prevent the skin from drying.

Nowadays, because of various reasons such as artificial temperature control for cooling/heating due to changes of the environment or life patterns, skin stresses caused by various stresses from social lives and environmental pollution, frequent washing due to makeups, and natural skin aging, etc. due to increase in age, the water content of the stratum corneum decreases, so that the skin becomes dry, the skin surface becomes rough, and the skin becomes loose and looks rusty due to lack of moisture, etc. Thus, there is an increasing need for skin moisturizers. Further, excessive physical and chemical stimuli from the outside, ultraviolet rays, stress and nutritional deficiencies deteriorate the normal functions of the skin and accelerate such phenomena as loss of elasticity, keratinization, wrinkle formation, etc. In particular, the dermis-epidermis border is severely damaged by ultraviolet rays.

Interleukin 1 is classified as a senescence-associated secretory phenotype (SASP). In particular, interleukin 1b (IL-1b) is known to be involved in cellular senescence as a representative aging marker (see non-patent document 5). It is also known that IL-1 b and PTGS2 are involved in skin inflammation, and that XDH indicates oxidative stress induced by prolonged exposure to an irritation source and thus is an indicator of extrinsic skin aging (see non-patent document 1). With regard to the skin inflammation, it is also known that the expression level of PTGS2 (also known as COX-2) is increased in skin cells exposed to an irritation source (see non-patent document 6). It is also known that XDH (xanthine dehydrogenase), which is an indicator of oxidative stress, is involved in skin aging.

IL-36G is known as a useful biomarker in psoriasis, etc. caused by the weakening of skin barrier (see non-patent document 2). In addition, S100A7 is known as an indicator of atopic dermatitis and psoriasis caused by the impairment of skin barrier function (see non-patent document 3). LCE3D is also known as an indicator of psoriasis risk genes (see non-patent document 4). These literatures are incorporated herein by reference in their entirety.

### REFERENCES OF RELATED ART

### Non-Patent Documents

(Non-patent document 1) Kim, H. J., et al, “Transcriptome analysis of airborne PM_{2.5}-induced detrimental effects on human keratinocytes”, Toxicology Letters 273, 26-35, 2017.
(Non-patent document 2) AM D'Erme et al, “IL-36c (IL-1F9) Is a Biomarker for Psoriasis Skin Lesions”, Journal of Investigative Dermatology, Volume 135, 2015.
(Non-patent document 3) Son et al, “S100A7 (psoriasin) inhibits human epidermal differentiation by enhanced IL-6 secretion through ljB/NF-jB signalling”, Experimental Dermatology, John Wiley & Sons A/S, 2016.
(Non-patent document 4) Bergboer et al, “Psoriasis Risk Genes of the Late Cornified Envelope-3 Group Are Distinctly Expressed Compared with Genes of Other LCE Groups”, The American Journal of Pathology, Vol. 178, No. 4, April 2011.
(Non-patent document 5) Jean-Philippe Coppe, et al, “The Senescence-Associated Secretory Phenotype: The Dark Side of Tumor Suppression”, Annual Review of Pathology: Mechanisms of Disease, volume 5, 2010.
(Non-patent document 6) Natalia D Magnani, et al, “Skin Damage Mechanisms Related to Airborne Particulate Matter Exposure”, Toxicological Sciences, 149(1), 2016, 227-236.

### DETAILED DESCRIPTION OF THE INVENTION

### Technical Problem

The present inventors have found that fine dust has harmful effects on skin, which affect the expression of skin cell genes, thereby causing symptoms such as damages of skin cells, etc.

Accordingly, in an aspect, the present disclosure is directed to providing a composition for treating skin cell damage caused by fine dust.

### Technical Solution

In an aspect, the present disclosure provides, as a composition containing a fermented tea extract as an active ingredient, a composition for treating skin damage caused by fine dust, which regulates the expression level of one or more selected from a group consisting of IL-1B (NM_000576), IL-36G (NM_019618), S100A7 (NM_002963), LCE3D (NM_032563), PTGS2 (NM_000963) and XDH (NM_000379), which are genes in skin cells the expression level of which is affected by fine dust, to a normal level.

### Advantageous Effects

In an aspect, by using a composition for treating skin damage caused by fine dust, skin cell damage can be treated by returning the expression level of the genes changed by fine dust to a normal level.

In an aspect, by using a composition for enhancing skin barrier provided by the present disclosure, skin cell damage may be reduced by returning the expression level of the genes changed by an irritation source which weakens skin barrier to a normal level.

In an aspect, by using an antioxidant composition, an antiaging composition or an antiinflammatory composition provided by the present disclosure, skin cell damage may be reduced by returning the expression level of the genes changed by inflammation or aging to a normal level.

### BRIEF DESCRIPTION OF DRAWINGS

FIG. 1 shows the effect of treatment with an irritation source on cell viability. ADSP (Asian dust storm particle) refers to Asian dust, PM10 refers to fine dust having a particle size of 10 μm, and PM2.5 refers to fine dust having a particle size of 2.5 μm.

FIG. 2A shows that the mRNA expression level of the IL-36G gene is increased in skin cells irradiated by PM2.5 fine dust, and is returned to a normal level by treatment with fermented tea.

FIG. 2B shows that the mRNA expression level of the IL-1B gene is increased in skin cells irradiated by PM2.5 fine dust, and is returned to a normal level by treatment with fermented tea.

FIG. 2C shows that the mRNA expression level of the PTGS2 gene is increased in skin cells irradiated by PM2.5 fine dust, and is returned to a normal level by treatment with fermented tea.

FIG. 2D shows that the mRNA expression level of the LCE3D gene is increased in skin cells irradiated by PM2.5 fine dust, and is returned to a normal level by treatment with fermented tea.

FIG. 2E shows that the mRNA expression level of the XDH gene is increased in skin cells irradiated by PM2.5 fine dust, and is returned to a normal level by treatment with fermented tea.

FIG. 2F shows that the mRNA expression level of the S100A7 gene is increased in skin cells irradiated by PM2.5 fine dust, and is returned to a normal level by treatment with fermented tea.

FIG. 3A shows a result of analyzing the ingredients of fermented tea through liquid chromatography-mass spectrometry (LC-MS). The chromatogram of raw TIC is shown.

FIG. 3B shows a result of analyzing the ingredients of fermented tea through liquid chromatography-mass spectrometry (LC-MS). The chromatogram of quinic acid in the raw material is shown.

FIG. 3C shows a result of analyzing the ingredients of fermented tea through liquid chromatography-mass spectrometry (LC-MS). The chromatogram of standard quinic acid is shown.

FIG. 3d shows a result of analyzing the ingredients of fermented tea through liquid chromatography-mass spectrometry (LC-MS). The chromatogram of the molecular weight of quinic acid is shown.

### DETAILED DESCRIPTION OF THE PREFERRED EMBODIMENTS

Hereinafter, the present disclosure is described in detail.

In an aspect of the present disclosure, a composition for treating skin damage caused by fine dust may comprise a fermented tea extract as an active ingredient.

In an aspect of the present disclosure, a composition for enhancing skin barrier may comprise a fermented tea extract as an active ingredient.

In the present disclosure, the “fermented tea” refers to tea in fermented state, specifically post-fermented tea.

In the present disclosure, the “post-fermented tea” refers to tea which has been fermented and aged using microbes under appropriate conditions of moisture, temperature, etc.

In an aspect, the post-fermented tea used in the present disclosure may be a pulverization product of the post-fermented tea itself or a dried pulverization product of the post-fermented tea, although not being limited thereto.

In an aspect of the present disclosure, the fermented tea may comprise quinic acid.

In the composition for enhancing skin barrier according to an exemplary embodiment of the present disclosure, the tea may be green tea. In another aspect of the present disclosure, the tea may be fermented green tea.

In an aspect, the fermented tea may be one that has been fermented and aged.

In an aspect, the fermented tea may be obtained by naturally fermenting the leaf of tea having deactivated enzymes.

In an aspect of the present disclosure, the fermented tea extract may be prepared by extracting fermented tea with an extraction solvent.

In an aspect of the present disclosure, the fermented tea extract may be prepared by extracting fermented tea with water or an organic solvent. Specifically, it may be prepared by extracting fermented tea with one or more extraction solvent selected from a group consisting of water, a C₁-C₆ anhydrous or water-containing lower alcohol, acetone, butylene glycol, ethyl acetate, diethyl acetate, diethyl ether, benzene, chloroform and hexane.

In an aspect, the fermented tea extract may be extracted at room temperature.

In an aspect, the fermented tea extract may be obtained by extracting with the extraction solvent and then further performing one or more of evaporation, filtration, concentration, separation and drying. In particular, the fermented tea extract may be subjected to one or more filtration process. In an exemplary embodiment, it is subjected to two filtration processes.

In an exemplary embodiment, the separation process may include a centrifugation process.

Specifically, the extraction may be performed using one or more of a polar solvent including water, a C₁-C₆ anhydrous or water-containing lower alcohol, acetone and butylene glycol and a low-polarity solvent including ethyl acetate, diethyl acetate, diethyl ether, benzene, chloroform and hexane as a solvent.

More specifically, the solvent may be a 50-90% ethanol aqueous solution and may be a 60-80% or 65-75% ethanol aqueous solution. When the solvent is a 50-90% ethanol aqueous solution, the active ingredient can be effectively extracted from fermented tea. In one embodiment, the solvent may be an about 70% ethanol aqueous solution.

In an aspect, the extract may be concentrated under reduced pressure at an appropriate temperature in a distillation apparatus equipped with a cooling condenser after the extraction.

However, the fermented tea extract according to the present disclosure can be obtained by extraction according to a common method in the art, and the extraction method is not limited to the above-described methods.

In an aspect of the present disclosure, the composition may contain 0.000001-30 wt % of the fermented tea extract based on the total weight of the composition. When the content thereof is 0.000001-30 wt %, the fermented tea extract exhibits an excellent effect of treating skin damage caused by fine dust and enhancing skin barrier, as well as superior antioxidant, antiaging and antiinflammatory effects.

Specifically, the content may be 0.0000001 wt % or more, 0.0000005 wt % or more, 0.0000007 wt % or more, 0.0000009 wt % or more, 0.000001 wt % or more, 0.000002 wt % or more, 0.000004 wt % or more, 0.000006 wt % or more, 0.000008 wt % or more, 0.00001 wt % or more, 0.00003 wt % or more, 0.00005 wt % or more, 0.00007 wt % or more, 0.00009 wt % or more, 0.0001 wt % or more, 0.0003 wt % or more, 0.0005 wt % or more, 0.0007 wt % or more, 0.0009 wt % or more, 0.001 wt % or more, 0.01 wt % or more, 0.1 wt % or more, 1 wt % or more, 3 wt % or more, 5 wt % or more, 7 wt % or more, 9 wt % or more, 10 wt % or more, 13 wt % or more, 15 wt % or more, 17 wt % or more, 19 wt % or more, 21 wt % or more, 23 wt % or more, 25 wt % or more, 27 wt % or more, 29 wt % or more, 30 wt % or more or 31 wt % or more, and 32 wt % or less, 31 wt % or less, 30 wt % or less, 29 wt % or less, 28 wt % or less, 26 wt % or less, 24 wt % or less, 22 wt % or less, 20 wt % or less, 18 wt % or less, 16 wt % or less, 14 wt % or less, 12 wt % or less, 10 wt % or less, 9 wt % or less, 8 wt % or less, 6 wt % or less, 4 wt % or less, 2 wt % or less, 1 wt % or less, 0.1 wt % or less, 0.09 wt % or less, 0.04 wt % or less, 0.01 wt % or less, 0.006 wt % or less, 0.001 wt % or less, 0.0009 wt % or less, 0.0007 wt % or less, 0.00005 wt % or less, 0.00003 wt % or less, 0.00001 wt % or less, 0.000009 wt % or less, 0.000007 wt % or less, 0.000005 wt % or less, 0.000003 wt % or less, 0.000001 wt % or less, 0.0000009 wt % or less, 0.0000007 wt % or less, 0.0000005 wt % or less, 0.0000003 wt % or less, 0.0000002 wt % or less, 0.0000001 wt % or less or 0.00000009 wt % or less, although not being limited thereto.

In another aspect, the composition is for treating skin damage caused by fine dust.

The term “fine dust” used in the present disclosure refers to very small particulate matter invisible to human eyes, which floats or flutters in the atmosphere for a long time. It may refer to dust with a particle diameter of 10 μm or less. In particular, the particulate matter having a particle diameter of 2.5 μm or less is called “ultrafine dust”. In the present disclosure, the term “fine dust” is intended to include “ultrafine dust”.

In the present disclosure, the term “treatment” refers to effective protection of skin cells from irritation and inhibition, prevention or restoration (recovery) of change in the expression level of a specific gene by the irritation.

In an aspect, the present disclosure provides a composition for inhibiting skin damage caused by fine dust by regulating the expression level of specific genes in skin cells damaged by fine dust to a normal level.

In an aspect, the composition may be applied to keratinocytes.

Specifically, in the present disclosure, the genes in skin cells the expression level of which is affected by fine dust include IL-1B (NM_000576), IL-36G (NM_019618), S100A7 (NM_002963), LCE3D (NM_032563), PTGS2 (NM_000963), XDH (NM_000379), etc. Since the IL IL-1B (NM_000576), IL-36G (NM_019618), S100A7 (NM_002963), LCE3D (NM_032563), PTGS2 (NM_000963) and XDH (NM_000379) are the genes the expression level of which is increased by fine dust, skin cell damage can be inhibited by reducing the expression level of these genes to a normal level.

The genes used in the present disclosure, the expression level of which is increased by fine dust, are listed in Table 1. Table 1 shows the genes the expression level of which is increased by fine dust. In the table, the “name” is the GeneBank accession ID of the NCBI, the “gene symbol” is the official gene symbol, and the “gene title” is the name of each gene. They are described in the non-patent document 1.

TABLE 1
Increased genes

| Name | Gene symbol | Gene title |
|---|---|---|
| NM_002963 | S100A7 | S100 calcium-binding protein A7 |
| NM_032563 | LCE3D | Late cornified envelope 3D |
| NM_019618 | IL36G | Interleukin 36, gamma |
| NM_000576 | IL1B | Interleukin 1, beta |
| NM_000963 | PTGS2 | Cyclooxygenase-2 (COX-2) |
| NM_000379 | XDH | Xanthine dehydrogenase |

In another aspect, the present disclosure provides a use of the composition of the present disclosure for enhancing skin barrier.

In another aspect, the present disclosure provides a method for enhancing skin barrier in a subject. The method may comprise administering an effective amount of a composition comprising a fermented tea extract as an active ingredient to a subject in need thereof.

In another aspect, the present disclosure provides a use of a fermented tea extract in the manufacture of a composition for enhancing skin barrier.

In another aspect, the present disclosure provides a fermented tea extract for enhancing skin barrier.

In another aspect, the present disclosure provides a composition for enhancing skin barrier by regulating the expression level of specific genes in skin cells damaged by skin barrier-weakening irritation to a normal level.

In an aspect, the composition may be applied to keratinocytes.

Specifically, in an aspect, the genes in skin cells the expression level of which is affected by the weakening of skin barrier include S100A7 (NM_002963), IL-36G (NM_019618), LCE3D (NM_032563), etc. Since the S100A7 (NM_002963), IL-36G (NM_019618) and LCE3D (NM_032563) are the genes the expression level of which is increased due to the skin barrier-weakening irritation, the skin barrier may be enhanced by reducing the expression level of these genes to a normal level.

The genes used in the present disclosure, the expression level of which is increased by the skin barrier-weakening irritation, are listed in Table 2. In the table, the “name” is the GeneBank accession ID of the NCBI, the “gene symbol” is the official gene symbol, and the “gene title” is the name of each gene.

TABLE 2
Increased genes

| Name | Gene symbol | Gene title |
|---|---|---|
| NM_002963 | S100A7 | S100 calcium-binding protein A7 |
| NM_019618 | IL36G | Interleukin 36, gamma |
| NM_032563 | LCE3D | Late cornified envelope 3D |

In another aspect, the present disclosure relates to antioxidant, antiaging and anti-inflammatory uses of the composition of the present disclosure.

In an aspect, the present disclosure provides a composition for inhibiting oxidation, inflammation or aging by regulating the expression level of specific genes in skin cells damaged by oxidation, inflammation or aging irritation to a normal level.

In an aspect, the composition may be applied to keratinocytes.

Specifically, in an aspect of the present disclosure, the genes in skin cells the expression level of which is affected by oxidation, inflammation or aging irritation include IL-1B (NM_000576), PTGS2 (NM_000963), XDH (NM_000379), etc. Since the IL-1B (NM_000576), PTGS2 (NM_000963) and XDH (NM_000379) are the genes cells the expression level of which is increased by oxidation, inflammation or aging irritation, the oxidation, inflammation or aging of skin cells can be inhibited by reducing the expression level of these genes to a normal level.

The genes used in the present disclosure the expression level of which is increased by oxidation, inflammation or aging irritation are described in Table 3. In the table, the “name” is the GeneBank accession ID of the NCBI, the “gene symbol” is the official gene symbol, and the “gene title” is the name of each gene.

TABLE 3
Increased genes

| Name | Gene symbol | Gene title |
|---|---|---|
| NM_000576 | IL1B | Interleukin 1, beta |
| NM_000963 | PTGS2 | Cyclooxygenase-2 (COX-2) |
| NM_000379 | XDH | Xanthine dehydrogenase |

The expression level of these genes or proteins may be analyzed using various analysis methods known in the art, such as microarray, PCR, NGS (next-generation sequencing), western blot, northern blot, ELISA, radioimmunoassay, radioimmunodiffusion, immunohistochemical staining, immunoprecipitation assay, etc.

In an aspect of the present disclosure, the composition may be a cosmetic composition, a pharmaceutical composition or a health functional food composition.

For example, the cosmetic composition may be a cosmetic such as a cream, a lotion, a toner, a cleanser, a face cleanser, a soap, a beauty care solution, etc.

In an aspect, the cosmetic to which the composition containing a fermented tea extract of the present disclosure is added may be in the form of a solution, an emulsion, a viscous mixture, etc.

That is to say, in an aspect, the formulation of the cosmetic of the present disclosure is not particularly limited. For example, the formulation may be an emulsion, a cream, a toner, an essence, a pack, a gel, a powder, a makeup base, a foundation, a lotion, an ointment, a patch, a cosmetic solution, a cleansing foam, a cleansing cream, a cleansing water, a body lotion, a body cream, a body oil, a body essence, a shampoo, a rinse, a body cleanser, a soap, a hair dye, a spray, etc.

Ingredients other than the fermented tea extract may be selected and added to the cosmetic composition of each formulation without difficulty by those skilled in the art in consideration of the formulation or purpose of use.

In addition, in an aspect, the cosmetic of the present disclosure may contain one selected from a group consisting of a water-soluble vitamin, an oil-soluble vitamin, a polypeptide, a polysaccharide, a sphingolipid and a seaweed extract.

In addition, in an aspect, the cosmetic of the present disclosure may contain ingredients that are generally used in cosmetics in addition to the essential ingredient, if necessary.

Examples of the additional ingredients include oils and fats, moisturizers, emollients, surfactants, organic and inorganic pigments, organic powders, UV absorbers, preservatives, sterilizers, antioxidants, plant extracts, pH adjusters, alcohols, colorants, fragrance, blood circulation stimulants, skin coolers, antiperspirants, purified water, etc.

However, the ingredients that may be contained in the cosmetic are not limited thereto. Also, the amount of any of the ingredients may be determined within a range not negatively affecting the purpose and effect of the present disclosure.

In an aspect, the pharmaceutical composition comprising a fermented tea extract of the present disclosure may further contain a suitable carrier, excipient and diluent commonly used for preparation of pharmaceutical compositions.

The pharmaceutical composition containing the fermented tea extract may be formulated into any form suitable for pharmaceutical preparations, including oral formulations such as a tablet, a capsule, a powder, a syrup, etc. and agents for external application to the skin such as an ointment, a gel, a cream, a patch, a spray, etc. according to common methods.

In general, it is to be understood that the actual dosage of the active ingredient administered by the pharmaceutical composition should be determined in light of various relevant factors such as the severity of the symptom, the selected administration route, the age, gender, body weight and health condition of a subject, etc. In general, the dosage of the active ingredient may be 0.0001-3000 mg/kg/day, for example, 10-500 mg/kg/day.

In the health functional food composition according to an aspect of the present disclosure, the health food may refer to a food prepared from nutrients which are likely to be deficient in normal diets or raw materials or ingredients (functional raw materials) with functions useful for the human body, and which maintain and improve health by maintaining the normal function of the human body or activating physiological functions, although not being limited thereto. The health food may be prepared and processed into the form of a tablet, a capsule, a powder, a granule, a liquid, a pill, etc. However, the formulation is not limited thereto, and it may be prepared and processed into any form under the law.

Specifically, a health beverage composition is not particularly limited in ingredients other than the above-described compound contained in the predetermined ratio as an essential ingredient. It may contain various flavoring agents or natural carbohydrates as additional ingredients as in common beverages. Examples of the natural carbohydrates are conventional sugars such as a monosaccharide, a polysaccharide, a cyclodextrin, etc. and sugar alcohols such as xylitol, sorbitol, erythritol, etc. Also, natural flavoring agents (thaumatin, stevia extract (e.g., rebaudioside A, glycyrrhizin, etc.)) and synthetic flavoring agents (e.g., saccharin, aspartame, etc.) may be used as the flavoring agent.

In general, it is to be understood that the actual dosage of the active ingredient administered by the health functional food composition should be determined in light of various relevant factors such as the severity of the symptom, the selected administration route, the age, gender, body weight and health condition of a subject, etc. In general, the dosage of the active ingredient may be 0.0001-1000 mg/kg/day, for example, 0.02-6 mg/kg/day.

Hereinafter, the constitution and effect of the present disclosure will be described in more detail with reference to examples. However, the following examples are provided for illustrative purposes only to facilitate understanding of the present disclosure, and the scope of the present disclosure is not limited thereto.

### [Example 1] Preparation of Fermented Tea

1-1 Fermentation Step

Fermented tea was prepared by naturally fermenting the dried leaves of green tea under a high-temperature, high-humidity condition, such that the water content in the green tea leaves was 30-50%. The prepared green tea leaves was fermented at 45° C. for 6 weeks.

1-2 Aging Step

1-1 Fermented tea was prepared by aging the fermented tea fermented in the fermentation step in a Jeju onggi for 50 days.

### [Example 2] Analysis of Ingredients of Fermented Tea

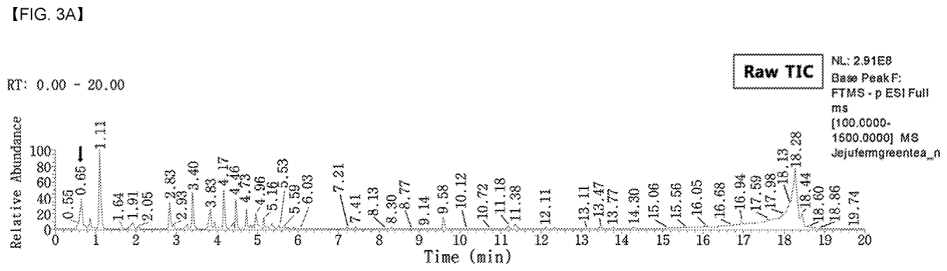

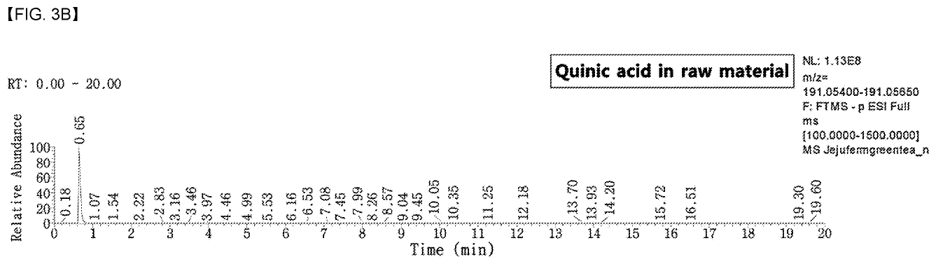

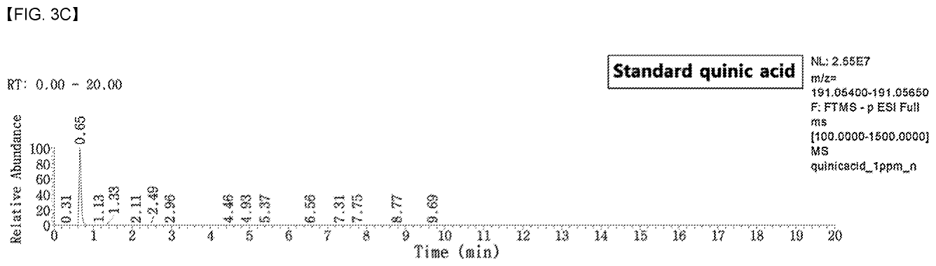

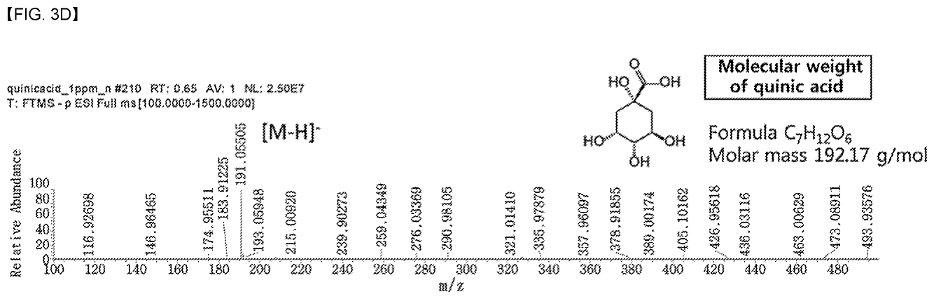

The ingredients of the fermented tea prepared in Example 1 were analyzed using by liquid chromatography-mass spectrometry (Q Exactive High Resolution LC-MS; Thermo). As can be seen from the chromatogram of FIG. 3, standard quinic acid was detected at 0.65 minute and had a theoretical m/z value of 191.05556 in the negative ion mode. For the fermented tea, a peak was detected at the same retention time of 0.65 minute when ion extraction was conducted in the molecular weight range from 191.05400 to 191.05650 in the negative ion mode TIC. The peak had an m/z value of 191.05505, which was equal to the theoretical value for quinic acid, 191.05556, up to the fourth decimal point with an error smaller than 2.6 ppm. Accordingly, the peak of the material at 0.65 minute can be contributed to quinic acid.

### [Example 3] Preparation of Fermented Tea Extract

The fermented tea of Example 1 was extracted at room temperature using an extraction solvent obtained by mixing purified water and ethanol at a ratio of 3:7, i.e., 70% ethanol, as an extraction solvent. After the extraction at room temperature, primary filtration was performed to remove the solid material contained in the extract. Then, the extract was concentrated to remove ethanol, followed by separation and purification. Then, the resultant was subjected to centrifugation and secondary filtration, and then dried to obtain a fermented tea extract.

### [Example 4] Preparation of Skin Barrier-Weakening Irritation Source

As a skin barrier-weakening irritation source, fine dust was collected using a low-volume air sampler (Sensidyne, Gillian, Fla., USA). The filter and denuder of a filter pack were replaced around 10:00 am on each measurement day, and the sample was collected for about 24 hours. Fine dust was collected daily for 28 days in an area downwind from Seoul, Korea (on the rooftop of a six-story building (Hankuk University of Foreign Studies, Center for International Studies, Residence Hall), Cheoin-gu, Yongin-si, Gyeonggi-do). Sampling time was recorded by checking the time while a vacuum pump was operated using a timer. Sampling rate, which was set to 16.7 L/min, was measured when the sampling was started and finished using a flow meter (Model 4143, TSI Inc.). A Teflon filter loaded into the filter pack was weighed before and after the sampling. Before weighing the Teflon filter, it was settled for 24 hours in a desiccator (Nikko, Japan) of 40% relative humidity. The weight was measured twice using an electronic balance (DVG215CD, Ohaus) to the five digits to the right of the decimal point and then averaged. Also, after the sampling, the filter was weighed twice after settlement in a desiccator for 24 hours. Mass concentration was calculated from the weight measured before the sampling. The fine dust was extracted as follows. The Teflon filter was soaked in 1 mL of ethanol. After adding 14 mL of DW so that the water level reached the aerosol sampling surface of the filter, followed by capping, extraction was conducted for 30 minutes by sonication. After completely removing water from the filter in a desiccator for 48 hours to minimize error, the weight of the filter before and after the extraction was measured using a high-precision balance (Mettler Toledo Company) which can measure up to 0.1 mg.

### [Example 5] Culturing of (Human Normal) Keratinocytes

Keratinocytes (human normal epidermal keratinocytes) purchased from Lonza, Inc. (Walkersville, Md., USA) were subcultured and then cultured in a CO₂ incubator under the condition of 37° C. and 5% CO₂. The cells were cultured according to Lonza's guidelines. The KGM-2 Bullet kit CC-3107 in which the KGM-2 Bullet kit CC-4152 (BPE (bovine pituitary extract), human epidermal growth factor (hEGF), insulin, hydrocortisone, transferrin, epinephrine and gentamycin sulfate+amphotericin-B (GA-1000)) was added to 500 mL of a KBM-2 (KBMTM-2, CC-3103) medium was used.

### [Example 6] Treatment of (Normal Human) Keratinocytes with Fine Dust and Measurement of Cytotoxicity

In order to investigate the effect of treatment with fine dust on cytotoxicity, MTT assay was performed with the (normal human) keratinocytes according to the method of Mossman et al. (J. Immunol. Methods, 65, 55-63, 1983).

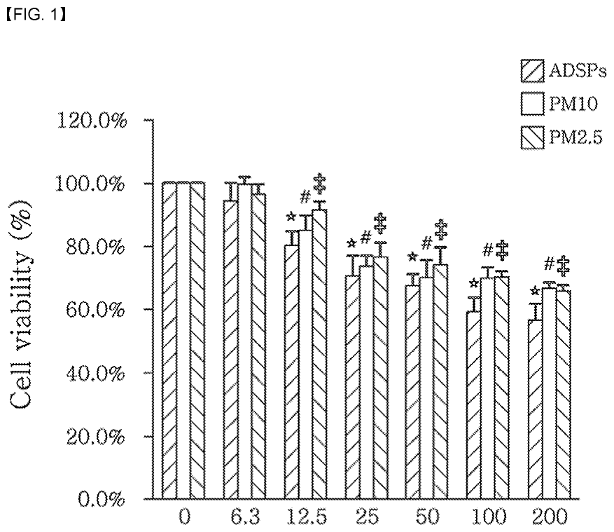

Specifically, a 24-well plate was used. The fine dust obtained in Example 4, with a diameter of 2.5 μm, was dispersed in purified water to prepare a fine dust dispersion. Then, the fine dust dispersion was applied to the cells cultured under the conditions of Example 5, with 2.5×10⁵ cells per well, followed by culturing for 24 hours. Then, the cells were mixed with 5 mg/mL of MTT (3-4,5-dimethylthiazol-2,5-diphenyltetrazolium bromide) and further cultured at 37° C. for 3 hours. The medium was then removed and the formazan crystal formed was dissolved in 500 μL of DMSO. The lysate was aliquoted to a 96-well plate and the OD value was measured at 540 nm. The measurement result is shown in FIG. 1.

As shown in FIG. 1, the concentration achieving 80% cell viability (1020) for cytotoxicity caused by the dispersion obtained by dispersing fine dust with a diameter of 2.5 μm or smaller was 12.5 μg/mL.

### [Example 7] Investigation of Change in Genes Due to Fine Dust by Next-Generation Sequencing

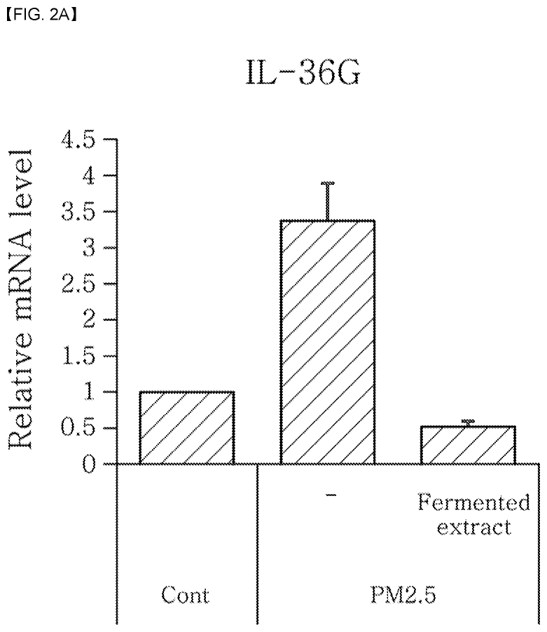

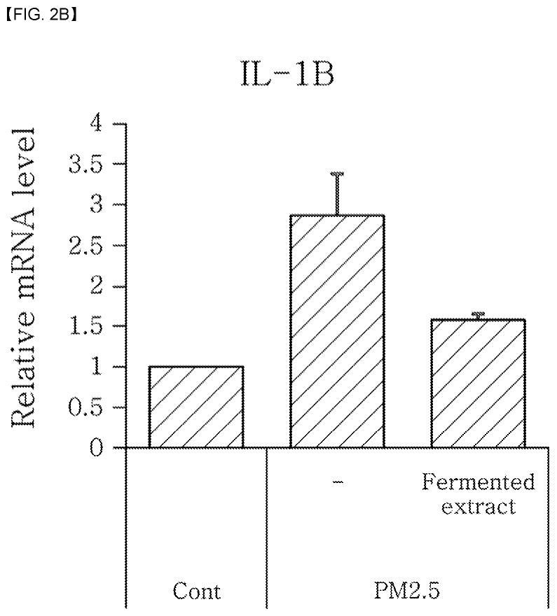

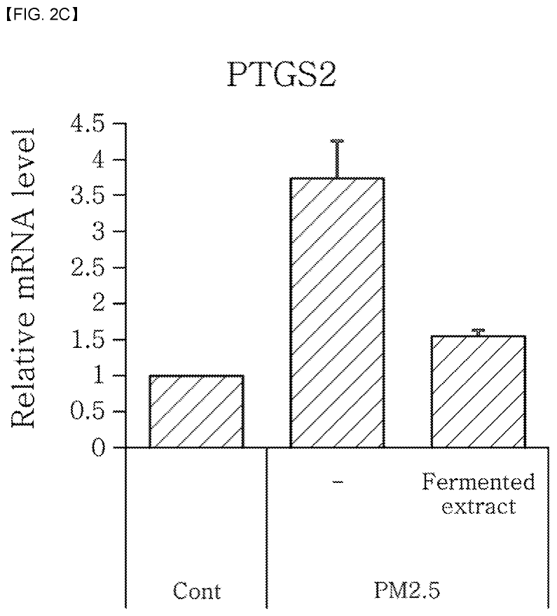

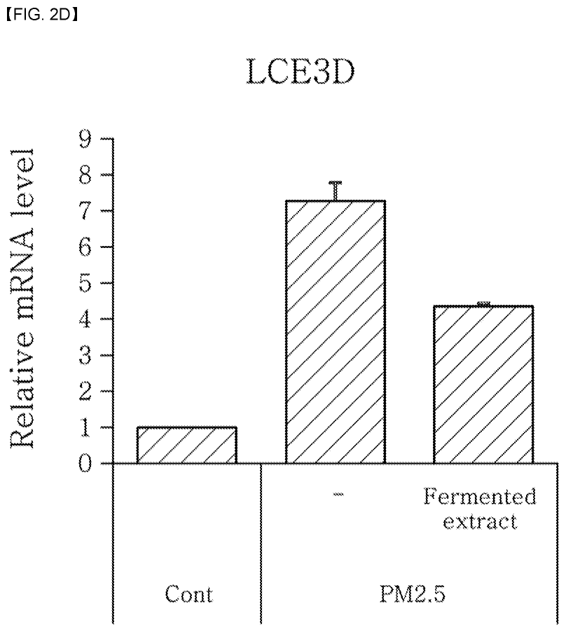

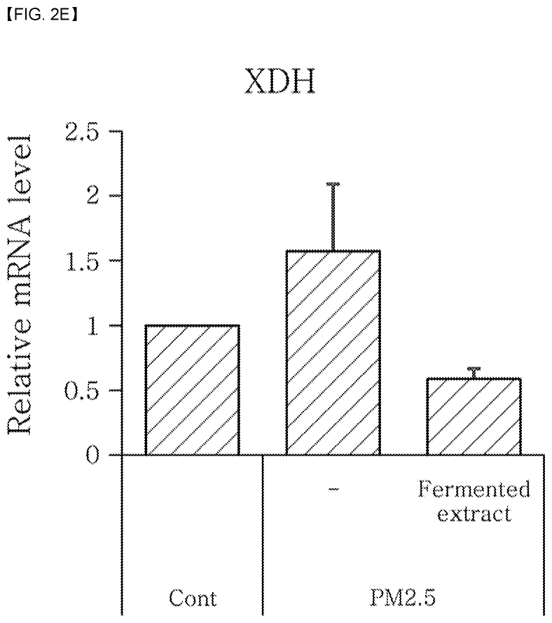

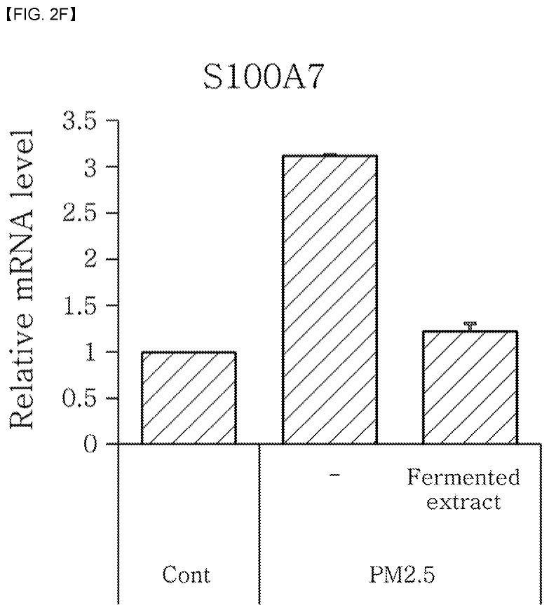

For RNA-base sequence data processing and analysis, reference was made to the general analysis technique developed by Trapnell et al. (2012). The RNA-seq data quality was determined using FastQC (http://www.bioinformatics.babraham.ac.uk/projects/fastqc/). The base and adapter sequences with low accuracy were removed using FASTX (http://hannonlab.cshl.edu/fastx_toolkit/). Then, alignment was performed using Tophat (Trapnell et al., 2009) and a human genome (hg19), and the amount of data of each sample was determined using EVER-seq renamed to RSeQC (Wang et al., 2012). In addition, the expression level of transcripts was quantified using Cufflinks, and transcription levels were compared between the sample treated with the fine dust dispersion and a normal sample (Trapnell et al., 2010). A stringent cut-off of 2.0-fold change, with the FDR-adjusted p-value<0.05, was used to determine the gene that showed significant difference in expression upon treatment with the dispersion of fine dust with a diameter of 2.5 μm. The measurement result is shown in Table 3 and FIGS. 2A-2F.

TABLE 4
Increased genes

| Name | Gene symbol | Fold change |
|---|---|---|
| NM_000576 | IL1B | 10.7 |
| NM_019618 | IL36G | 7.1 |
| NM_002963 | S100A7 | 23.8 |
| NM_032563 | LCE3D | 12.0 |
| NM_000963 | PTGS2 | 3.7 |
| NM_000379 | XDH | 2.4 |

### [Example 8] Real-Time RT-PCR

The normal human keratinocytes cultured in Example 5 were treated with the fine dust having a diameter of 2.5 μm extracted in Example 4, with 12.5 μg per 1 mL of the cell culture medium. Then, the relative mRNA expression level was measured using the primers (Applied Biosystems TaqMan® primers) of the genes described in Table 5. The fermented tea extract prepared in Example 3 was used.

TABLE 5
Increased genes

| Name | Gene symbol | TaqMan ® primers |
|---|---|---|
| NM_000576 | IL1B | Hs01555410_m1 |
| NM_019618 | IL36G | Hs00219742_m1 |
| NM_002963 | S100A7 | Hs00161488_m1 |
| NM_032563 | LCE3D | Hs00754375_s1 |
| NM_000963 | PTGS2 | Hs00153133_m1 |
| NM_000379 | XDH | Hs00166010_m1 |

The medium was treated with 20 ppm of the fermented tea extract. After 24 hours, the culture solution was removed and the cells were washed with 2 mL of phosphate-buffered saline (PBS). Then, RNA was isolated from the cells using a Trizol reagent (Invitrogen, Carlsbad, Calif., USA). The isolated RNA was further purified with the QIAGEN RNeasy kit (QIAGEN, Valencia, Calif.). Then, the quality of the RNA was determined using the Agilent 2100 BioAnalyzer (Agilent Technologies, Santa Clara, Calif., USA). cDNA was synthesized from the RNA using the Superscript Reverse Transcriptase (RT) kit (Invitrogen, Carlsbad, Calif.). The cDNA was quantitatively analyzed by real time-reverse transcription polymerase chain reaction (Q-RT-PCR) using the primers shown in Table 5. The change in the expression pattern of genes was evaluated by real-time PCR using the TaqMan gene expression assay kit (Applied Biosystems, Foster City, Calif.). The result is shown in FIGS. 2A-2F. Both of the Q-RT-PCR and the real-time PCR were performed according to the standard protocols distributed by Life Technologies. Specifically, 40 cycles of 95° C. for 20 seconds, 95° C. for 3 seconds and 60° C. for 30 seconds were performed.

FIGS. 2A-2F show that there exist genes the expression level of which is increased or decreased in the skin cells irritated by fine dust. Also, it was found that the expression level of the interleukin 1 beta (IL-1B), interleukin 36 gamma (IL-36G), S100 calcium-binding protein A7 (S100A7), late cornified envelope 3D (LCE3D), prostaglandin-endoperoxide synthase 2 (PTGS2) and xanthine dehydrogenase (XDH) genes is decreased by treatment with the fermented tea extract.

Therefore, it was found that the fermented tea extract effectively protects skin cells from irritation by fine dust and inhibits or prevents the change in the expression level of the above-described specific genes due to the irritation, thereby returning the expression level to a normal level. In addition, it was found that the fermented tea effectively protects skin cells from skin cell damage caused by skin barrier-weakening irritation and inhibits or prevents the change in the expression level of the above-described specific genes due to the irritation, thereby returning the expression level to a normal level. In addition, it was found that the fermented tea effectively protects skin cells from skin cell damage caused by oxidation, inflammation or aging irritation and inhibits or prevents the change in the expression level of the above-described specific genes due to the irritation, thereby returning the expression level to a normal level.

Hereinafter, formulation examples of the compositions according to the present disclosure will be described. However, the cosmetic composition, pharmaceutical composition and health functional food composition may be formulated into various other forms. These examples are for illustrative purposes only and are not intended to limit the scope of the present disclosure.

### [Formulation Example 1] Tablet

100 mg of the fermented tea extract according to the present disclosure, 400 mg of lactose, 400 mg of corn starch and 2 mg of magnesium stearate were mixed and subjected to a tableting process according to a conventional method for preparing tablets to prepare a tablet.

TABLE 6

| Ingredients | Contents (mg) |
|---|---|
| Fermented tea extract | 100 |
| Lactose | 400 |
| Corn starch | 400 |
| Magnesium stearate | 2 |

### [Formulation Example 2] Capsule

100 mg of the fermented tea extract according the present disclosure, 400 mg of lactose, 400 mg of corn starch and 2 mg of magnesium stearate were mixed and filled in a gelatin capsule according to a conventional method for preparing capsules to prepare a capsule.

TABLE 7

| Ingredients | Contents (mg) |
|---|---|
| Fermented tea extract | 100 |
| Lactose | 400 |
| Corn starch | 400 |
| Magnesium stearate | 2 |

### [Formulation Example 3] Granule

50 mg of the fermented tea extract according to the present disclosure, 250 mg of anhydrous crystalline glucose and 550 mg of starch were mixed and formulated into granules using a fluidized-bed granulator. The granules were then filled in a pouch.

TABLE 8

| Ingredients | Contents (mg) |
|---|---|
| Fermented tea extract | 50 |
| Anhydrous crystalline glucose | 250 |
| Starch | 550 |

### [Formulation Example 4] Soap

TABLE 9

| Ingredients | Contents (%) |
|---|---|
| Fermented tea extract | 5.00 |
| Oil and fat | q.s. |
| Sodium hydroxide | q.s. |
| Sodium chloride | q.s. |
| Fragrance | q.s. |
| Purified water | balance |

### [Formulation Example 5] Lotion

TABLE 10

| Ingredients | Contents (%) |
|---|---|
| Fermented tea extract | 5.00 |
| Magnesium L-ascorbic acid-2-phosphate | 1.00 |
| Water-soluble collagen (1% aqueous solution) | 1.00 |
| Sodium citrate | 0.10 |
| Citric acid | 0.05 |
| Licorice extract | 0.20 |
| 1,3-Butylene glycol | 3.00 |
| Purified water | balance |

### [Formulation Example 6] Cream

TABLE 11

| Ingredients | Contents (%) |
|---|---|
| Fermented tea extract | 3.00 |
| Polyethylene glycol monostearate | 2.00 |
| Self-emulsifying glycerin monostearate | 5.00 |
| Cetyl alcohol | 4.00 |
| Squalene | 6.00 |
| Glyceryl tri(2-ethylhexanoate) | 6.00 |
| Sphingoglycolipid | 1.00 |
| 1,3-Butylene glycol | 7.00 |
| Purified water | balance |

### [Formulation Example 7] Ointment

TABLE 12

| Ingredients | Contents (%) |
|---|---|
| Fermented tea extract | 5.00 |
| Polyvinyl alcohol | 13.00 |
| Magnesium L-ascorbic acid-2-phosphate | 1.00 |
| Lauroyl hydroxyproline | 1.00 |
| Water-soluble collagen (1% aqueous solution) | 2.00 |
| 1,3-Butylene glycol | 3.00 |
| Ethanol | 5.00 |
| Purified water | balance |

### [Formulation Example 8] Beauty Care Solution

TABLE 13

| Ingredients | Contents (%) |
|---|---|
| Fermented tea extract | 3.00 |
| Hydroxyethylene cellulose (2% aqueous solution) | 12.00 |
| Xanthan gum (2% aqueous solution) | 2.00 |
| 1,3-Butylene glycol | 6.00 |
| Concentrated glycerin | 4.00 |
| Sodium hyaluronate (1% aqueous solution) | 2.00 |
| Purified water | balance |

### [Formulation Example 9] Health Food

TABLE 14

| Ingredients | Contents | |
|---|---|---|
| Fermented tea extract | 2 | mg |
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Nicotinamide | 1.7 | mg |
| Folic acid | 50 | μg |
| Calcium pantothenate | 0.5 | mg |
| Ferrous sulfate | 1.75 | mg |
| Zinc oxide | 0.82 | mg |
| Magnesium carbonate | 25.3 | mg |
| Potassium phosphate monobasic | 15 | mg |
| Calcium phosphate dibasic | 55 | mg |
| Potassium citrate | 90 | mg |
| Calcium carbonate | 100 | mg |
| Magnesium chloride | 24.8 | mg |

### [Formulation Example 10] Health Drink

TABLE 15

| Ingredients | Contents | |
|---|---|---|
| Fermented tea extract | 50 | mg |
| Citric acid | 1000 | mg |
| Oligosaccharide | 100 | g |
| Taurine | 1 | g |

| | |
|---|---|
| Purified water | balance |

1. A method for treating skin cell damage caused by fine dust, comprising administering an effective amount of a composition comprising a fermented tea extract as an active ingredient to a subject in need thereof.

2. A method for enhancing skin barrier, comprising administering an effective amount of a composition comprising a fermented tea extract as an active ingredient to a subject in need thereof.

3. A method for antioxidation, antiaging, or antiinflammatory, comprising administering an effective amount of a composition comprising a fermented tea extract as an active ingredient to a subject in need thereof.

4. (canceled)

5. (canceled)

6. The method according to claim 1, wherein the fermented tea is obtained by naturally fermenting leaf of tea that has been subjected to an enzyme-deactivation treatment.

7. The method according to claim 1, wherein the fermented tea extract is comprised in an amount of 0.000001-30 wt % based on the total weight of the composition.

8. The method according to claim 1, wherein the fermented tea extract is extracted with one or more extraction solvent selected from a group consisting of water, a C₁-C₆ anhydrous or water-containing lower alcohol, acetone, butylene glycol, ethyl acetate, diethyl acetate, diethyl ether, benzene, chloroform, and hexane.

9. The method according to claim 1, wherein the composition inhibits the expression of one or more selected from a group consisting of IL-1B (NM_000576), IL-36G (NM_019618), S100A7 (NM_002963), LCE3D (NM_032563), PTGS2 (NM_000963) and XDH (NM_000379).

10. (canceled)

11. (canceled)

12. The method according to claim 1, wherein the composition is applied to keratinocytes.

13. The method according to claim 1, wherein the fine dust has a particle size of 2.5 μm or smaller.

14. The method according to claim 1, wherein the fermented tea extract is administered at a dosage of 10-500 mg/kg/day.

15. The method according to claim 1, wherein the composition is a cosmetic composition.

16. The method according to claim 1, wherein the composition is a health functional food composition.

17. The method according to claim 2, wherein the fermented tea is obtained by naturally fermenting leaf of tea that has been subjected to an enzyme-deactivation treatment.

18. The method according to claim 2, wherein the fermented tea extract is comprised in an amount of 0.000001-30 wt % based on the total weight of the composition.

19. The method according to claim 2, wherein the fermented tea extract is extracted with one or more extraction solvent selected from a group consisting of water, a C₁-C₆ anhydrous or water-containing lower alcohol, acetone, butylene glycol, ethyl acetate, diethyl acetate, diethyl ether, benzene, chloroform, and hexane.

20. The method according to claim 2, wherein the composition inhibits the expression of one or more selected from a group consisting of IL-36G (NM_019618), S100A7 (NM_002963) and LCE3D (NM_032563).

21. The method according to claim 2, wherein the composition is applied to keratinocytes.

22. The method according to claim 2, wherein the fermented tea extract is administered at a dosage of 10-500 mg/kg/day.

23. The method according to claim 2, wherein the composition is a cosmetic composition.

24. The method according to claim 2, wherein the composition is a health functional food composition.

25. The method according to claim 3, wherein the fermented tea is obtained by naturally fermenting leaf of tea that has been subjected to an enzyme-deactivation treatment.

26. The method according to claim 3, wherein the fermented tea extract is comprised in an amount of 0.000001-30 wt % based on the total weight of the composition.

27. The method according to claim 3, wherein the fermented tea extract is extracted with one or more extraction solvent selected from a group consisting of water, a C₁-C₆ anhydrous or water-containing lower alcohol, acetone, butylene glycol, ethyl acetate, diethyl acetate, diethyl ether, benzene, chloroform, and hexane.

28. The method according to claim 3, wherein the composition inhibits the expression of one or more selected from a group consisting of IL-1B (NM_000576), PTGS2 (NM_000963) and XDH (NM_000379).

29. The method according to claim 3, wherein the composition is applied to keratinocytes.

30. The method according to claim 3, wherein the fermented tea extract is administered at a dosage of 10-500 mg/kg/day.

31. The method according to claim 3, wherein the composition is a cosmetic composition.

32. The method according to claim 3, wherein the composition is a health functional food composition.
